(19) United States
(12) Patent Application Publication
(10) Pub. No.: US 2026/0284311 A1
(43) Pub. Date: Sep. 24, 2026

(54) A pause mechanism for a drive assembly of a medicament delivery device

(71) Applicant: SHL Medical AG, Zug (CH)

(72) Inventors: Sebastian Karlsson, Stigtomta (SE); Núria De Santiago Giner, Zug (CH); René Zander, Zug (CH); Urs Jann, Zug (CH)

(21) Appl. No.: 19/473,841

(22) PCT Filed: Apr. 8, 2024
(86) PCT No.: PCT/EP2024/059467

§ 371 Date: Oct. 9, 2025

(30) Foreign Application Priority Data
Apr. 21, 2023 (EP) 23169254.2

(51) Int. Cl. A61M 5/20; A61M 5/315; A61M 5/32

(52) U.S. Cl. CPC A61M 5/2033; A61M 5/31528; A61M 5/31568; A61M 5/3243; A61M 2005/2073

(57) ABSTRACT

The present disclosure provides a pause mechanism for a drive assembly of a medicament delivery device. The drive assembly includes a driver configured to be rotationally driven by a power source and an actuator sleeve. The pause mechanism includes a resilient member operably connected to the actuator sleeve and is configured to be tensioned by the actuator sleeve when the actuator sleeve is moved from the first position to the second position. When the actuator sleeve is moved from the second position to the first position by the resilient member the blocking structure is re-engaged with the driver.

### TECHNICAL FIELD

The present disclosure generally relates to a pause mechanism for a drive assembly of a medicament delivery device, particularly a pause mechanism for a drive assembly having a rotational driver.

### BACKGROUND

Medicament delivery devices may be designed to automatically perform a medicament administration operation, i.e. to expel a medicament. Such an operation may be triggered by the user when interacting with the medicament delivery device. The user may for example initiate a medicament administration operation by moving a needle cover extending from the housing into the housing or by pushing a button provided on the medicament delivery device. This movement triggers internal components to cause an automatic medicament expulsion. The medicament expulsion may for example be provided by means of one or more springs and/or by means of an internal motor. One example of medicament delivery devices of this type is auto-injectors.

Furthermore, most of the self-administration medicament delivery devices are required to lock the devices when the user removes the devices from the medicament delivery site so that the used medicament delivery members cannot be accessed. Thus, if the user removes a medicament delivery device too early, the device will be locked, and the user cannot get the residual medicament. For some medicaments, it might be crucial to know the exact intake of the user. However, most of the self-administration medicament delivery devices are required to lock the devices from exposing the medicament delivery members, e.g., needles, meaning that even if the devices are locked, the medicament will still be injected into the air. As a result, the user or anyone else cannot observe from used devices to understand what exact dose the user has been injected into the user's body. It might be crucial for the self-administration medicament delivery devices as the devices are designed for the user to carry out the medicament delivery operation without supervision. Thus, there is a need to have a mechanism to have the used devices be capable of providing information about what exact intake of the users.

### SUMMARY

The invention is defined by the appended claims, to which reference should now be made. In the present disclosure, when the term “distal direction” is used, this refers to the direction pointing away from the dose delivery site during use of the medicament delivery device. When the term “distal part/end” is used, this refers to the part/end of the delivery device, or the parts/ends of the members thereof, which under use of the medicament delivery device is/are located furthest away from the dose delivery site. Correspondingly, when the term “proximal direction” is used, this refers to the direction pointing towards the dose delivery site during use of the medicament delivery device. When the term “proximal part/end” is used, this refers to the part/end of the delivery device, or the parts/ends of the members thereof, which under use of the medicament delivery device is/are located closest to the dose delivery site.

Further, the term “longitudinal”, “longitudinally”, “axially” or “axial” refer to a direction extending from the proximal end to the distal end, typically along the device or components thereof in the direction of the longest extension of the device and/or component.

Similarly, the terms “transverse”, “transversal” and “transversally” refer to a direction generally perpendicular to the longitudinal direction.

Further, the terms “circumference”, “circumferential”, or “circumferentially” refer to a circumference or a circumferential direction relative to an axis, typically a central axis extending in the direction of the longest extension of the device and/or component. Similarly, “radial” or “radially” refer to a direction extending radially relative to the axis, and “rotation”, “rotational” and “rotationally” refer to rotation relative to the axis.

There is hence provided a drive assembly of a medicament delivery device, the drive assembly comprising a driver configured to be rotationally driven by a power source, an actuator sleeve, a blocking structure positioned radially between the actuator sleeve and the driver when the actuator sleeve is in the first position, wherein the blocking structure is configured to engage with the driver when the actuator sleeve is in the first position such that the rotation of the driver relative to the blocking structure is prevented, wherein when the actuator sleeve is moved from the first position towards the second position the actuator sleeve is configured to release the blocking structure from the driver, enabling the driver to rotate. The drive assembly comprises a resilient member operably connected to the actuator sleeve and is configured to be tensioned by the actuator sleeve when the actuator sleeve is moved from the first position to the second position. When the actuator sleeve is moved from the second position to the first position by the resilient member the blocking structure is re-engaged with the driver.

As a result, a pause mechanism of the drive assembly can be provided. Namely, after the driver is disengaged from the blocking structure to be rotated by the power source, the blocking structure can re-engage with the driver again to stop the rotation of the driver and hold the driver against the power source.

Thus, activation of a medicament delivery device may be controlled by means of the position of the actuator sleeve.

According to one embodiment, the drive assembly comprises a button movable between a pre-activation position and an activation position. In this example, the actuator sleeve is operably connected to the button such that when the button moves from the pre-activation position to the activation position, the movement of the button causes the actuator sleeve moves from the first position to the second position.

According to one embodiment, the button is directly connected with the actuator sleeve such that the button is configured to move the actuator sleeve from the first position to the second position when the button is moved by a user of the medicament delivery device.

Alternatively, according to another embodiment, the drive assembly comprises a delivery member cover configured to be moved axially relative to the driver, between an extended position and a retracted position, wherein the delivery member cover is configured to move the actuator sleeve from a first position to a second position when the delivery member cover is moved from the extended position to the retracted position.

According to one embodiment, the delivery member cover is directly connected with the actuator sleeve such that the delivery member cover is configured to move the actuator sleeve from the first position to the second position when the delivery member cover is moved by a user of the medicament delivery device.

According to one embodiment, the delivery member cover comprises a proximal tubular portion to surround the medicament delivery member. A proximal end of the proximal tubular portion is configured to be in contact with a medicament delivery site when the medicament delivery operation starts.

The driver may for example be spring driven or configured to be driven by an electric motor. In other words, the power source that is configured to rotationally drive the driver can be a torsion spring or a motor.

The drive assembly is for a medicament delivery device. The medicament delivery device may for example be an auto-injector.

According to one embodiment, the actuator sleeve is axially immovable relative to the delivery member cover.

According to one embodiment, the resilient member is longitudinally extending between the actuator sleeve and the driver.

According to one embodiment, the resilient member is compressed when the actuator sleeve is moved from the first position to the second position.

According to one embodiment, the pause mechanism comprises a residual dose indication.

According to one embodiment, the residual dose indication comprises an axially extending scale indication configured to be mechanically connected to the driver and at least partially observable from outside of a housing of the medicament delivery device such that the residual dose of the medicament of the medicament delivery device can be shown by the extending scale indication to a user of the medicament delivery device when the driver is stopped rotating.

According to one embodiment, the residual dose indication comprises an indication drum having the axially extending scale indication.

According to one embodiment, the indication drum comprises a thread engaged with a counter thread of the driver such that the rotation of the driver converts to an axial movement of the indication drum via the engagement between the thread and the counter thread.

According to one embodiment, the drive assembly comprises a plunger rod operably engaged with the driver such that the driver moves the plunger rod axially when the driver is rotated.

According to one embodiment, the plunger rod is rotationally engaged with the driver such that the plunger rod is rotated by the driver when the driver is rotated.

According to one embodiment, the plunger rod comprises a plunger rod head and a plunger rod body axially extending from the plunger rod head.

According to one embodiment, the plunger rod head is configured to be operably engaged with a medicament container of the medicament delivery device.

According to one embodiment, the residual dose indication is provided with the plunger rod body comprising an axially extending scale indication.

According to one embodiment, the axial extending scale indication is positioned on a housing of the medicament delivery device.

According to one embodiment, the axial extending scale indication is positioned adjacent to and along a main window of the housing of the medicament delivery device.

According to one embodiment, the axially extending scale indication is formed by at least one of color marks, numerals, textures, letters, geometric marks, and a combination thereof.

According to another aspect of the invention, a drive assembly comprises the pause mechanism as mentioned above is provided.

According to one embodiment, the drive mechanism comprises a driver configured to be rotationally driven by a power source, an actuator sleeve, a blocking structure positioned radially between the actuator sleeve and the driver when the actuator sleeve is in a first position, wherein the blocking structure is configured to engage with the driver when the actuator sleeve is in the first position such that the rotation of the driver relative to the blocking structure is prevented, and wherein when the actuator sleeve is moved from the first position towards a second position the actuator sleeve is configured to release the blocking structure from the driver, enabling the driver to rotate.

According to one embodiment, the drive assembly comprises a button operably connected to the actuator sleeve. The button is configured to be moved by a user of the medicament delivery device from a pre-activation position to an activation position. The movement of the button from the pre-activation position to the activation position causes the actuator sleeve to move from the first position to the second position.

According to one embodiment, the drive assembly comprises a delivery member cover configured to be moved axially relative to the driver between an extended position and a retracted position.

According to one embodiment, the delivery member cover is configured to move the actuator sleeve from a first position to a second position when the delivery member cover is moved from the extended position to the retracted position.

According to one embodiment, the delivery member cover comprises a proximal tubular portion to surround the medicament delivery member. A proximal end of the proximal tubular portion is configured to be in contact with a medicament delivery site when the medicament delivery operation starts.

According to one embodiment, the driver comprises a counter-blocking structure radially extending towards the blocking structure. The blocking structure is configured to engage with the counter-blocking structure of the driver when the actuator sleeve is in the first position.

According to one embodiment, the counter-blocking structure of the driver comprises protrusions radially extending towards the blocking structure; wherein the blocking structure is configured to engage with the protrusions when the actuator sleeve is in the first position.

According to one embodiment, the blocking structure comprises a protrusion extending radially towards the driver.

According to one embodiment, the counter-blocking structure is a set of ratchet teeth.

According to one embodiment, the drive assembly comprises a blocking member.

According to one embodiment, the blocking member is ring-shaped.

According to one embodiment, the blocking member comprises a flexible arm being radially flexible. The blocking structure is arranged on a surface of the flexible arm facing towards the driver.

According to one embodiment the actuator sleeve is configured to prevent the flexible arm from moving radially outwards when the actuator sleeve is in the first position.

According to one embodiment the actuator sleeve is configured to enable the flexible arm to flex radially outwards when the actuator sleeve is in the second position.

According to one embodiment, the actuator sleeve comprises a support wall. The flexible arm is positioned radially between the driver and the support wall of the actuator sleeve when the actuator sleeve is in the first position such that the flexible arm is prevented from flexing radially outward.

According to one embodiment, the driver is configured to provide a rotational force on the flexible arms, urging the flexible arms radially outwards.

According to one embodiment, the flexible arm comprises a surface facing towards the actuator sleeve in the direction of the longitudinal axis. The actuator sleeve comprises a counter surface facing towards the flexible arm in the direction of the longitudinal axis.

According to one embodiment, at least one of the surface and the counter surface is a chamfered surface such that when the counter surface of the actuator sleeve moves along the surface of the flexible arm, the flexible arm is bent radially towards the drive.

According to one embodiment, the flexible arm comprises a free end. The surface of the flexible arm facing towards the actuator sleeve is located at the free end of the flexible arm.

According to one embodiment, the blocking structure is an integral part of the actuator sleeve extending from the actuator sleeve towards the driver.

According to one embodiment, the blocking structure is disengaged from the driver when the actuator sleeve is in the second position.

According to one embodiment, the driver is configured to provide a rotational force on the flexible arms, urging the flexible arm radially outwards.

Alternatively, or additionally, the flexible arm may be prestressed urging it to move radially outwards to its default position when the actuator sleeve is moved towards the second position.

According to one embodiment, the protrusion of the blocking structure comprises an inclined surface configured to cooperate with the radially outwards extending protrusions of the driver to urge the flexible arm radially outwards by rotation.

According to one embodiment, the radially outwards extending protrusions of the driver are the counter-blocking structure of the driver.

The inclined surface may be inclined relative to any radial plane extending through the central axis of the driver and the blocking member and/or relative to the corresponding surface of the protrusion of the driver configured to cooperate with the inclined surface.

According to one embodiment, the flexible arm is provided with a radial inner recess or groove configured to receive a respective one of the radially outwards extending protrusions of the driver.

According to one embodiment, each inner recess or groove has a sidewall which defines the inclined surface.

According to one embodiment, the actuator sleeve has a cut-out which is offset from the flexible arm in the first position and becomes aligned with the flexible arm in the second position, enabling the flexible arm to flex radially outwards.

According to one embodiment, the actuator sleeve has two cut-outs which are offset from the flexible arm in the first position and which become aligned with the flexible arm in the second position, enabling the flexible arm to flex radially outwards.

According to one embodiment, the two cut-outs of the actuator sleeve are symmetrically arranged in the actuator sleeve.

According to one embodiment, the actuator sleeve is axially immovable relative to the delivery member cover.

According to one embodiment, the actuator sleeve is snap-fitted to the delivery member cover.

According to one example, the actuator sleeve is the delivery member cover.

According to one embodiment, in the extended position, the actuator sleeve is in the first position and in the second delivery member position the actuator sleeve is in the second position.

According to one embodiment, the delivery member cover is configured to axially move the actuator sleeve from the first position to the second position when the delivery member cover is moved from the extended position to the second delivery member position.

According to one embodiment, the actuator sleeve is provided with a guide track configured to interact with the delivery member cover, causing linear movement of the delivery member cover to be transformed into rotational movement of the actuator sleeve, wherein the first position is a first rotational position and the second position is a second rotational position.

One embodiment comprises a plunger rod, wherein the driver is configured to receive the plunger rod.

Typically, the axial movement of a plunger rod may be triggered by the rotation of the driver. Rotation of the driver causes or contributes to the axial movement of the plunger rod.

The plunger rod may be provided with an external thread structure and the driver may be provided with an internal thread structure threadedly engaged with the external thread structure, enabling forward movement of the plunger rod when the driver is rotated.

Alternatively, an interface formed between the plunger rod and the driver has a non-circular cross-section observed along the central axis of the plunger rod, as a result, the driver is rotationally fixed to the plunger rod such that the plunger rod and the driver are rotated together. In the latter example, the plunger rod is threadedly engaged with a component that is configured to be rotationally immovable to the medicament container of the medicament delivery device. The component can be a part of the medicament delivery device or a part of the

There is according to another aspect of the present disclosure provides a medicament delivery device comprising the drive assembly as mentioned above.

According to one embodiment, the medicament delivery device is an autoinjector.

According to one embodiment, the medicament delivery device comprises a medicament container containing a medicament.

According to one embodiment, the medicament container is a syringe, a cartridge or a collapsible bag.

According to one embodiment, the medicament container is configured to contain 2 ml to 10 ml of medicament.

According to one embodiment, the medicament container is configured to contain 2.25 ml of medicament, 5 ml of medicament, and 5.5 ml of medicament.

According to one embodiment, the medicament delivery device comprises a medicament delivery member.

According to one embodiment, the medicament delivery member can be a needle, a soft cannular configured to be embedded under the skin of the user, or a spray nozzle.

The medicament delivery devices described herein can be used for the treatment and/or prophylaxis of one or more of many different types of disorders. Exemplary disorders include but are not limited to: rheumatoid arthritis, inflammatory bowel diseases (e.g. Crohn's disease and ulcerative colitis), hypercholesterolaemia, diabetes (e.g. type 2 diabetes), psoriasis, migraines, multiple sclerosis, anaemia, lupus, atopic dermatitis, asthma, nasal polyps, acute hypoglycaemia, obesity, anaphylaxis and allergies. Exemplary types of medicaments that could be included in the medicament delivery devices described herein include, but are not limited to, small molecules, hormones, cytokines, blood products, antibodies, antibody-medicament conjugates, bispecific antibodies, proteins, fusion proteins, peptibodies, polypeptides, pegylated proteins, protein fragments, protein analogues, protein variants, protein precursors, chimeric antigen receptor T cell therapies, cell or gene therapies, oncolytic viruses, or immunotherapies and/or protein derivatives. Exemplary medicaments that could be included in the medicament delivery devices described herein include, but are not limited to (with non-limiting examples of relevant disorders in brackets): etanercept (rheumatoid arthritis, inflammatory bowel diseases (e.g. Crohn's disease and ulcerative colitis)), evolocumab (hypercholesterolaemia), exenatide (type 2 diabetes), secukinumab (psoriasis), erenumab (migraines), alirocumab (rheumatoid arthritis), methotrexate (amethopterin) (rheumatoid arthritis), tocilizumab (rheumatoid arthritis), interferon beta-1a (multiple sclerosis), sumatriptan (migraines), adalimumab (rheumatoid arthritis), darbepoetin alfa (anaemia), belimumab (lupus), peginterferon beta-1a′ (multiple sclerosis), sarilumab (rheumatoid arthritis), semaglutide (type 2 diabetes, obesity), dupilumab (atopic dermatitis, asthma, nasal polyps, allergies), glucagon (acute hypoglycaemia), epinephrine (anaphylaxis), insulin (diabetes), atropine and vedolizumab (inflammatory bowel diseases (e.g. Crohn's disease and ulcerative colitis)), ipilimumab, nivolumab, pembrolizumab, atezolizumab, durvalumab, avelumab, cemiplimab, rituximab, trastuzumab, ado-trastuzumab emtansine, fam-trastuzumab deruxtecan-nxki, pertuzumab, transtuzumab-pertuzumab, alemtuzumab, belantamab mafodotin-blmf, bevacizumab, blinatumomab, brentuximab vedotin, cetuximab, daratumumab, elotuzumab, gemtuzumab ozogamicin, 90-Yttrium-ibritumomab tiuxetan, isatuximab, mogamulizumab, moxetumomab pasudotox, obinutuzumab, ofatumumab, olaratumab, panitumumab, polatuzumab vedotin, ramucirumab, sacituzumab govitecan, tafasitamab, or margetuximab. Pharmaceutical formulations including, but not limited to, any medicament described herein are also contemplated for use in the medicament delivery devices described herein, for example, pharmaceutical formulations comprising a medicament as listed herein (or a pharmaceutically acceptable salt of the medicament) and a pharmaceutically acceptable carrier. Pharmaceutical formulations comprising a medicament as listed herein (or a pharmaceutically acceptable salt of the medicament) may include one or more other active ingredients, or may be the only active ingredient present.

Exemplary medicaments that could be included in the medicament delivery devices described herein include, but are not limited to, an immuno-oncology or bio-oncology medications such as immune checkpoints, cytokines, chemokines, clusters of differentiation, interleukins, integrins, growth factors, enzymes, signaling proteins, pro-apoptotic proteins, anti-apoptotic proteins, T-cell receptors, B-cell receptors, or costimulatory proteins.

Exemplary medicaments that could be included in the medicament delivery devices described herein include, but are not limited to, those exhibiting a proposed mechanism of action, such as HER-2 receptor modulators, interleukin modulators, interferon modulators, CD38 modulators, CD22 modulators, CCR4 modulators, VEGF modulators, EGFR modulators, CD79b modulators, Trop-2 modulators, CD52 modulators, BCMA modulators, PDGFRA modulators, SLAMF7 modulators, PD-1/PD-L1 inhibitors/modulators, B-lymphocyte antigen CD19 inhibitors, B-lymphocyte antigen CD20 modulators, CD3 modulators, CTLA-4 inhibitors, TIM-3 modulators, VISTA modulators, INDO inhibitors, LAG3 (CD223) antagonists, CD276 antigen modulators, CD47 antagonists, CD30 modulators, CD73 modulators, CD66 modulators, CDw137 agonists, CD158 modulators, CD27 modulators, CD58 modulators, CD80 modulators, CD33 modulators, APRIL receptor modulators, HLA antigen modulators, EGFR modulators, B-lymphocyte cell adhesion molecule modulators, CDw123 modulators, Erbb2 tyrosine kinase receptor modulators, mesothelin modulators, HAVCR2 antagonists, NY-ESO-1 OX40 receptor agonist modulators, adenosine A2 receptors, ICOS modulators, CD40 modulators, TIL therapies, or TCR therapies.

Exemplary medicaments that could be included in the medicament delivery devices described herein include, but are not limited to, a multi-medication treatment regimen such as AC, Dose-Dense AC, TCH, GT, EC, TAC, TC, TCHP, CMF, FOLFOX, mFOLFOX6, mFOLFOX7, FOLFCIS, CapeOx, FLOT, DCF, FOLFIRI, FOLFIRINOX, FOLFOXIRI, IROX, CHOP, R-CHOP, RCHOP-21, Mini-CHOP, Maxi-CHOP, VR-CAP, Dose-Dense CHOP, EPOCH, Dose-Adjusted EPOCH, R-EPOCH, CODOX-M, IVAC, HyperCVAD, R-HyperCVAD, SC-EPOCH-RR, DHAP, ESHAP, GDP, ICE, MINE, CEPP, CDOP, GemOx, CEOP, CEPP, CHOEP, CHP, GCVP, DHAX, CALGB 8811, HIDAC, MOPAD, 7+3, 5+2, 7+4, MEC, CVP, RBAC500, DHA-Cis, DHA-Ca, DHA-Ox, RCVP, RCEPP, RCEOP, CMV, DDMVAC, GemFLP, ITP, VIDE, VDC, VAI, VDC-IE, MAP, PCV, FCR, FR, PCR, HDMP, OFAR, EMA/CO, EMA/EP, EP/EMA, TP/TE, BEP, TIP, VIP, TPEx, ABVD, BEACOPP, AVD, Mini-BEAM, IGEV, C-MOPP, GCD, GEMOX, CAV, DT-PACE, VTD-PACE, DCEP, ATG, VAC, VeIP, OFF, GTX, CAV, AD, MAID, AIM, VAC-IE, ADOC, or PE.

Exemplary medicaments that could be included in the medicament delivery devices described herein include, but are not limited to, those used for chemotherapy, such as an alkylating agent, plant alkaloid, antitumor antibiotic, antimetabolite, or topoisomerase inhibitor, enzyme, retinoid, or corticosteroid. Exemplary chemotherapy medicaments include, by way of example but not limitation, 5-fluorouracil, cisplatin, carboplatin, oxaliplatin, doxorubicin, daunorubicin, idarubicin, epirubicin, paclitaxel, docetaxel, cyclophosphamide, ifosfamide, azacitidine, decitabine, bendamustine, bleomycin, bortezomib, busulfan, cabazitaxel, carmustine, cladribine, cytarabine, dacarbazine, etoposide, fludarabine, gemcitabine, irinotecan, leucovorin, melphalan, methotrexate, pemetrexed, mitomycin, mitoxantrone, temsirolimus, topotecan, valrubicin, vincristine, vinblastine, or vinorelbine.

Furthermore, all terms used in the claims are to be interpreted according to their ordinary meaning in the technical field, unless explicitly defined otherwise herein. All references to “a/an/the element, apparatus, component, means, etc.” are to be interpreted openly as referring to at least one instance of the element, apparatus, component, means, etc., unless explicitly stated otherwise.

Generally, all terms used in the claims are to be interpreted according to their ordinary meaning in the technical field, unless explicitly defined otherwise herein. All references to “a/an/the element, apparatus, component, means, etc. are to be interpreted openly as referring to at least one instance of the element, apparatus, component, means, etc.”, unless explicitly stated otherwise.

### BRIEF DESCRIPTION OF THE DRAWINGS

The specific embodiments of the inventive concept will now be described, by way of example, with reference to the accompanying drawings, in which:

FIG. 1 is a perspective view of one example of a medicament delivery device;

FIG. 2 is a perspective view of components of the medicament delivery device of FIG. 1;

FIG. 3 is a perspective view of a drive assembly of the medicament delivery device of FIG. 1; FIGS. 4-6 are cross-section views and a perspective view of the drive assembly of FIG. 3;

FIG. 7 is a perspective view of another example of a medicament delivery device;

FIGS. 8-9 are perspective views of a drive assembly of the medicament delivery device of FIG. 7;

FIGS. 10-12 are perspective views of a pause mechanism of the drive assembly of FIG. 3 and FIGS. 8-9;

FIG. 13 is a perspective view of another example of a driver of the drive assembly of FIG. 3 and FIGS. 8-10;

FIG. 14 is a perspective view of another example of an actuator sleeve of the drive assembly of FIG. 3 and FIGS. 8-10;

FIGS. 15-17 are perspective views of different examples of residual dose indications of the pause mechanism of FIGS. 10-12.

FIGS. 18-19 are perspective views of a lockout lever of a delivery member cover lockout mechanism for the medicament delivery device of FIG. 1 and FIG. 7.

### DETAILED DESCRIPTION

The inventive concept will now be described more fully hereinafter with reference to the accompanying drawings, in which exemplifying embodiments are shown. The inventive concept may, however, be embodied in many different forms and should not be construed as limited to the embodiments set forth herein; rather, these embodiments are provided by way of example so that this disclosure will be thorough and complete, and will fully convey the scope of the inventive concept to those skilled in the art. Like numbers refer to elements throughout the description.

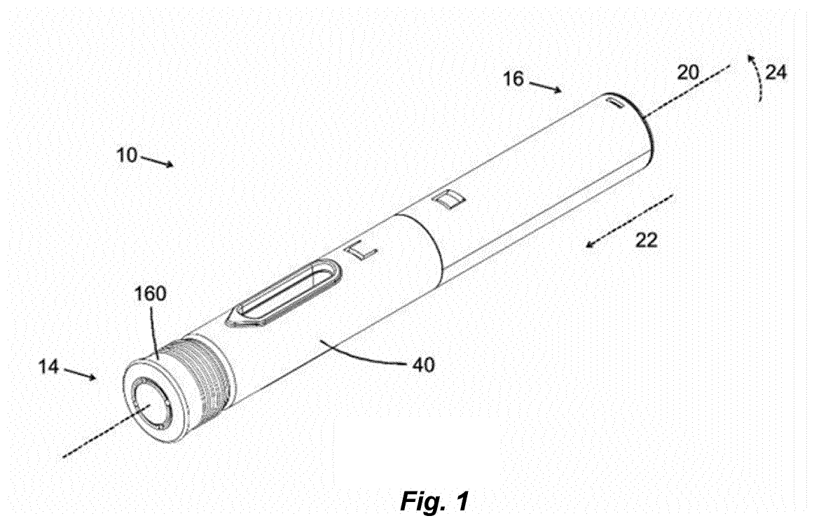

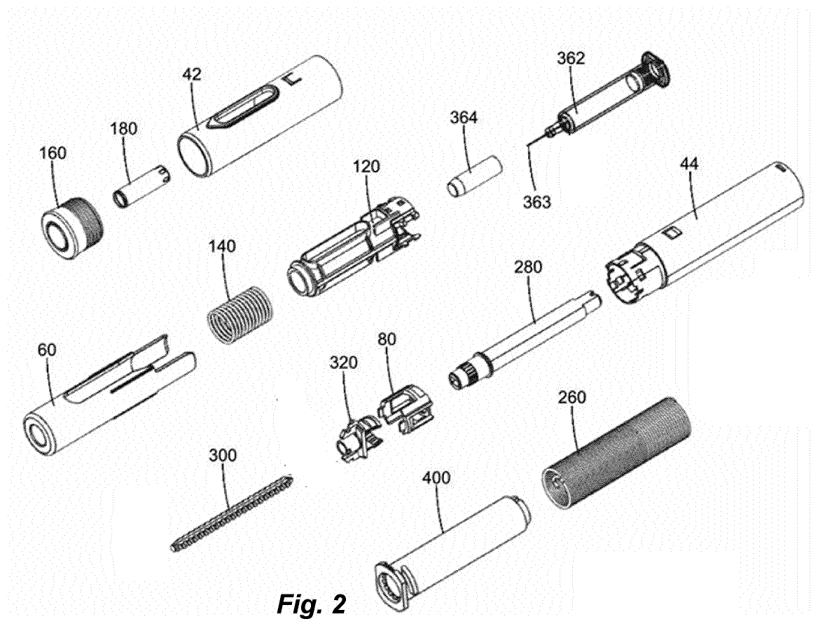

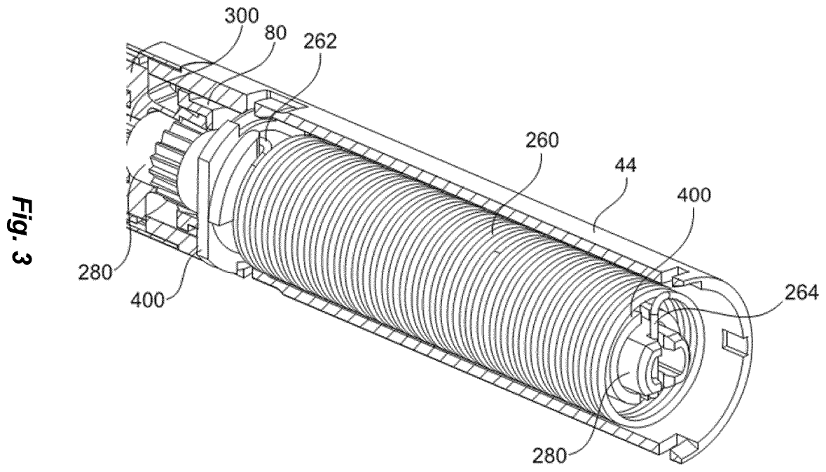

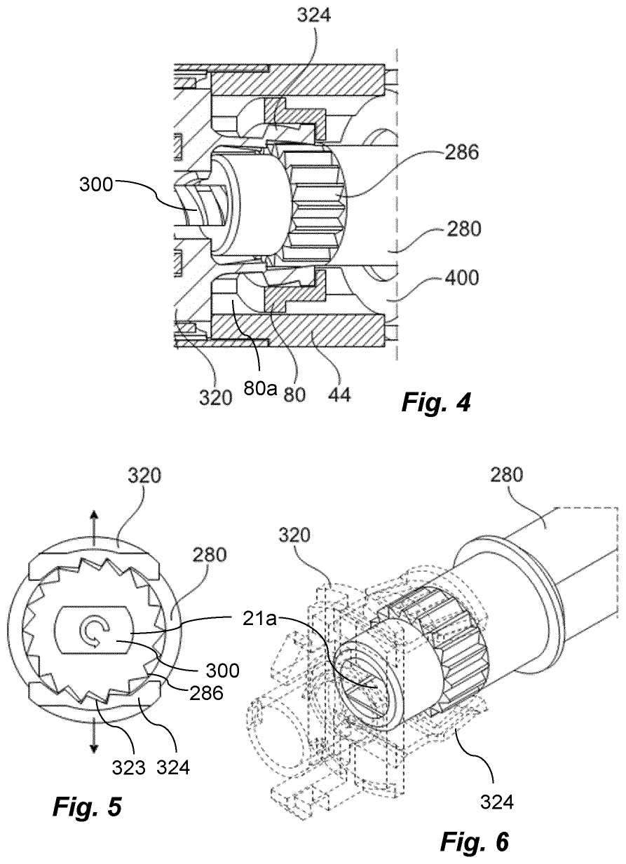

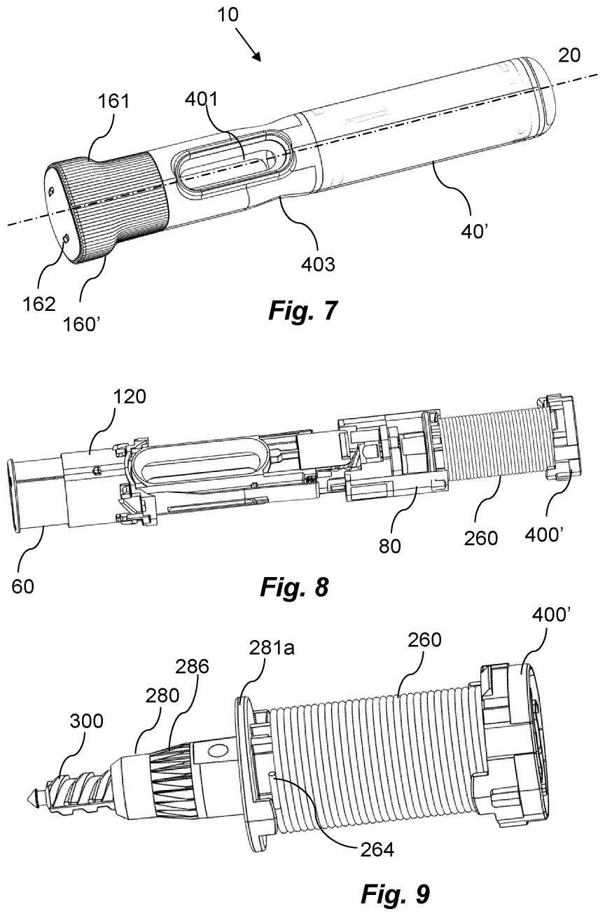

FIG. 1 and FIG. 7 show different examples of a medicament delivery device 10. The medicament delivery device 10 includes a housing 40; 40′. The housing 40; 40′ forms an elongated structure extending a longitudinal axis 20. The housing 40; 40′ includes a proximal housing part 42 and a distal housing part 44, as shown in FIG. 2. The housing 40 could alternatively be formed by a single housing 40′ part of by more than two housing parts.

The medicament delivery device 10 includes a removable cap 160; 160′. The cap 160; 160′ is attached to a proximal end 14 of the medicament delivery device 10. The medicament delivery device 10 has a distal end 16 opposite to the proximal end 14.

FIG. 2 shows an exploded view of the medicament delivery device 10. The medicament delivery device 10 comprises a delivery member cover 60 configured to be received by the housing 40. The delivery member cover 60 is configured to be moved between an extended position relative to the housing 40 and a retracted position. The delivery member cover 60 is configured to be biased towards the extended position. When the cap 160 has been removed, the delivery member cover 60 is arranged in the extended position. The medicament delivery device 10 comprises a first resilient member 140 configured to bias the delivery member cover 60 towards the extended position. The first resilient member 140 may for example be a spring such as a helical spring. In a preferred example, the delivery member cover 60 comprises a proximal tubular portion configured to surround a medicament delivery member of the medicament delivery device when the delivery member cover is in the extended position.

FIG. 7 shows another example of the medicament delivery device 10. The housing 40′ comprises a main window 401 such that the user can observe the medicament through the window. In a preferred example, the housing 40′ comprises a curved portion 403 such that the user can easily grip the housing 40′. The cap 160′, in a preferred example, comprises a curved portion 161 for being gripped by the user. The cap 160′ may also comprises one or more openings 162 to prevent potential suffocation if the cap 160′ is accidentally swallowed.

In a preferred example, the medicament delivery device 10 comprises a medicament container 362 provided with a medicament delivery member 363 such as a needle, and a medicament container holder 120 configured to receive the medicament container 362.

In another example, the medicament delivery device 10 comprises a delivery member shield 364 configured to protect the delivery member 363. The delivery member shield 364 may engage with the cap 160 such that the delivery member shield 364 is removed from the delivery member 363 together with the cap 160 when the cap 160 is removed.

Alternatively, the medicament container 362 can be a cartridge. In this example, instead of an integral delivery member 363 as shown in the previous example, the user of the medicament delivery device needs to attach a delivery member to the cartridge before use. The attachment of the delivery member to the cartridge can be done by the user of the medicament delivery device manually or by an automatic delivery mounting mechanism an operation of the medicament delivery device. In this example, the medicament delivery device doesn't need to have the delivery member shield 364.

The medicament delivery device 10 comprises an actuator sleeve 80 configured to engage with the delivery member cover 60. The actuator sleeve 80 is configured to be moved by the delivery member cover 60 such that when the delivery member cover 60 is moved from the extended position (as shown with arrow X1 in FIG. 11), the actuator sleeve 80 is moved from a first position in which the medicament delivery device 10 is in an initial state, as shown in FIG. 11 towards a second position in which the medicament delivery member is in an activation state, as shown in FIG. 11. The initial state is a pre-activation state of the medicament delivery device 10.

The delivery member cover 60 is configured to move the actuator sleeve 80 from the first position towards the second position. In one example, the actuator sleeve 80 and the delivery member cover 60 are made as separate components. In this example, the actuator sleeve 80 can be spaced apart from the delivery member cover in a direction of the longitudinal axis 20 with a predetermined distance. Thus, only when a user moves the delivery member cover with the predetermined distance, further movement of the delivery member cover can cause the actuator sleeve to move from the first position to the second position. Alternatively, the medicament delivery member cover is adjacent to the actuator sleeve. Alternatively, the actuator sleeve is immovable relative to the delivery member cover. For example, the actuator sleeve is snap-fitted to the delivery member cover; or the actuator sleeve and the delivery member cover could form a single integrated component.

The medicament delivery device 10 comprises a blocking structure 323 configured to releasably engage with the driver 280 such that the driver is releasably held by the blocking structure from rotating.

In one example, the medicament delivery device 10 comprises a blocking member 320 having the blocking structure 323, a plunger rod 300, a driver 280 and a power source 260. The blocking member 320, the driver 280, the delivery member cover 60 and the actuator sleeve 80 form a drive assembly for initiating a medicament delivery or medicament delivery operation of the medicament delivery device 10. Optionally, the drive assembly may comprises the plunger rod 300 of the medicament delivery device 10 and the housing of the medicament delivery device to provide a residual dose indication.

In a preferred example, the plunger rod 300 has an external surface provided with a plurality of external threads. The plunger rod 300 may have a non-circular cross-section. The plunger rod 300 may have a non-circular cross-sectional shape along its entire length or at least a majority of its length. The driver 280 has an opening 21a configured to receive the plunger rod 300. The opening 21a may have a non-circular cross-section. The opening 21a may for example be adapted to match the cross-sectional shape of the plunger rod 300, as shown in FIG. 5.

The driver 280 is configured to be connected to the power source 260. The driver 280 is configured to be rotated by the power source 260 in a circumferential direction 24. In a preferred example, the power source is a torsion spring. Alternatively, the power source is a motor.

In the example where the power source 260 is a torsion spring, one end of the torsion spring 260 is configured to be operably attached to the housing 40; 40′. For example, the one end 262 of the power source is attached to a wall of the housing directly, or the one end of the torsion spring 260 is attached to an inner housing 400; 400′ that is configured to be attached to the housing 40; 40′. The inner housing 400; 400′ can be a sleeve 400 or a distal cap 400′. The other end 264 of the torsion spring 260 is operably attached to the driver 280. In a preferred example, the torsion spring 260 is attached to the driver 280 directly so that the accumulated torque of the torsion spring 260 can be transferred to the driver 280 efficiently. In one example, as shown in FIG. 3, the other end 263 of the torsion spring 260 is attached to the distal end of the driver 280. In one example, cut-outs are arranged in a wall of the driver 280 for receiving the other end of the torsion spring 260. In another example, the other end 263 of the torsion spring 260 is attached to the proximal end or a middle portion of the driver, as shown in FIG. 9. In this example, the driver 280 comprises a flange 281 a. A groove is arranged in a wall of the flange 281a to receive the other end of the torsion spring 260.

The driver 280 is provided with a counter-blocking structure 286. In one example, the counter-blocking structures is a plurality of radially outwards extending protrusions 286. The blocking member 320 comprises a blocking structure 323; 323′ configured to releasably engage with the counter blocking structure 286 of the drive 280 in the circumferential direction 24. As a result, the driver 280 can be rotationally held against the torque from the torsion spring 260 by the blocking structure. The blocking structure 323; 323′ is positioned radially between the actuator sleeve 80 and the driver 280 when the actuator sleeve 80 is in the first position. In a preferred example, the blocking member 320 comprises flexible arms 324, the blocking structure 323 is extending from a surface of the flexible arm facing towards the driver 280, as shown in FIGS. 4-6.

In one example, the blocking structure is a protrusion. In a preferred example, the blocking structure is ratcheted teeth, and the counter-blocking structure is a counter-ratchet tooth matching with the blocking structure.

The disclosure below will be explained with the example where the protrusions as the counter-blocking structure of the driver. However, it should be noted that the counter-blocking structure and the blocking structure can be formed in any suitable structure that is rotationally mating to one another such that when the blocking structure is engaged with the counter-blocking structure, the rotation of the driver relative to the blocking structure is prevented. For example, the counter-blocking structure can be a cut-out or recess arranged in a wall of the driver. The cut-out or recess opens in a direction transverse to the longitudinal axis 20. In this example, the blocking structure can be a protrusion configured to be positioned in the cut-out or recess when the actuator sleeve is in the first position.

The blocking member 320 is configured to be rotationally locked relative to the housing 40. In the present example, the blocking member 320 is provided with engagement ribs extending radially outwards, configured to engage with an inner structure of the housing 40 to prevent rotation of the blocking member 320 relative to the housing 40.

The actuator sleeve 80 is in the first position arranged axially relative to the blocking member 320 such that the blocking structure of the arms 324 is held in engagement with the protrusions 286 by a support surface 80a of the actuator sleeve 80. In one example, the support surface 80a is the inner surface of the actuator sleeve 80, as shown in FIG. 4. The actuator sleeve 80 hence prevents the arms 324 from moving radially outwards from the protrusions 286 (as shown with arrows in FIG. 5). As a result, the blocking structure is engaged with the protrusions. In one example, the actuator sleeve 80 has a cut-out 81 which axially aligns with the arms 324 when the actuator sleeve 80 is moved towards the second position. The arms 324 are thereby able to move radially outwards from the protrusions 286. Alternatively, the actuator sleeve 80 is configured to move away from the arms in the longitudinal direction.

Once the arms are able to move radially outwards from the protrusion 286, the rotation of the driver 280 causes the protrusions 286 of the driver 280 to push the arms radially outwards. As a result, the driver 280 is thereby set free to rotate relative to the blocking member 320. The plunger rod 300 which has a matching cross-sectional shape with the driver 280, as shown in FIG. 5, will therefore rotate concurrently with the driver 280. In one example, a central opening of the blocking member is circular and provided with an internal thread configured to cooperate with the external threads of the plunger rod. Alternatively, the medicament delivery device further comprises an insert comprising an internal thread configured to cooperate with the external threads of the plunger rod. The thread engagement causes the plunger rod 300 to move forward through the opening 21a of the driver 280. The plunger rod 300 is thereby moved forward inside the medicament container 362. The plunger rod 300 is configured to act on a stopper 362a arranged in the medicament container 362.

In one example as shown in FIG. 6, the blocking member 320. The blocking member 320 is configured to receive a proximal portion of the driver 280. The blocking member 320 has a distal channel portion configured to receive the proximal portion of the driver 280. The proximal portion of the driver 280 comprises the protrusions 286. The distal channel portion is formed between the arms 324.

In the initial state of the medicament delivery device 10 the drive assembly is also in an initial state. The blocking structure 323 of the arms 324 of the blocking member 320 are in engagement with the protrusions 286 of the driver 280. This prevents the rotation of the driver 280, which is torsionally biased by the power source 260.

When a medicament administration operation is to be performed, the user will remove the cap 160 from the delivery member cover 60. The user will then place the proximal end of the delivery member cover 60 towards the injection site. As the user pushes the medicament delivery device 10 towards the site of injection, the delivery member cover 60 is moved into the housing 40 from the extended position towards the retracted position. In a preferred example, the first position of the delivery member cover 60 is the extended position and the second position of the delivery member cover 60 is the retracted position.

When the delivery member cover 60 is moved towards the distal end 1b of the medicament delivery device 10, i.e. towards the retracted position, the delivery member cover 60 moves the actuator sleeve 80 from the first position to the second position. As a result, the arms 324 are set free and are able to flex radially outwards, as shown in FIG. 5 with arrows.

In one example, the delivery member cover 60 is adjacent to the actuator sleeve 80. Alternatively, the delivery member cover 60 is spaced apart from the actuator sleeve 80, as shown in FIGS. 8, 10 and 12, such that any accidental movement of the delivery member cover 60 from the extended position to the retracted position, e.g., caused by the drop of the medicament delivery device, will not cause the movement of the actuator sleeve 80.

When the medicament delivery device 10 is in the activation state, the drive assembly is also in the activation state. The activation state is a state in which the plunger rod 300 has been set to move forward further into the medicament container 362. As the delivery member cover 60 has been moved fully to the retracted position, the activator sleeve 80 has been also moved to the second position. The arms 324 have hence aligned with the cut-out 81 and are able to move radially outwards from the protrusions 286. As mentioned above, the arms 324 may for example be urged radially outwards by the protrusions 286 which provide a rotating force onto the arms 324. Alternatively, or additionally, the arms 324 may be arranged in a pre-tensioned state when the actuator sleeve 80 bears against the arms 324 and they may strive for flexing radially outwards to obtain their unstressed default positions. As a result, the blocking structure of the arms 324 will disengage from the protrusions once the activator sleeve 80 is moved to the second position.

As the driver 280 is set in rotation the plunger rod 300 is also set in rotation concurrently with the driver 280. As a result, the plunger rod 300 is thus moved in the forward direction further into the medicament container 362. The medicament is thereby expelled from the medicament container 362.

Furthermore, in another embodiment, the blocking structure 323′ is an integral part of the actuator sleeve extending from the actuator sleeve towards the driver 80′, as shown in FIG. 14. When the actuator sleeve 80′ is in the first position, the blocking structure 323′ is engaged with the protrusions 286′ of the driver 280; and when the actuator sleeve 80′ is in the second position, the blocking structure 323′ is disengaged from the driver 280.

It should be noted that, in some of the examples disclosed herein, the activator sleeve could be provided in the form of a rotator, which is configured to rotate when the delivery member cover is moved from the extended position towards the retracted position. The first position is in this case a first rotational position and the second position is a second rotational position. The actuator sleeve may thus be provided with a cut-out that aligns with the arms when the actuator sleeve is rotated towards the second position.

When the delivery member cover 60 is released from being pushed into the housing 40. The delivery member cover 60 is thereby moved from the retracted position towards the extended position. This situation typically occurs when the user decides not to continue the injection, e.g., the medicament has been injected or the user cannot tolerate further injection, and the medicament delivery device 10 is removed from the injection site.

Furthermore, the drive assembly can provide a pause mechanism. The pause mechanism is configured to stop the driver 280 from rotating when the user lifts the medicament delivery device from the medicament delivery site. Therefore, the medicament delivery operation will be stopped, and the residual medicament will not be further injected.

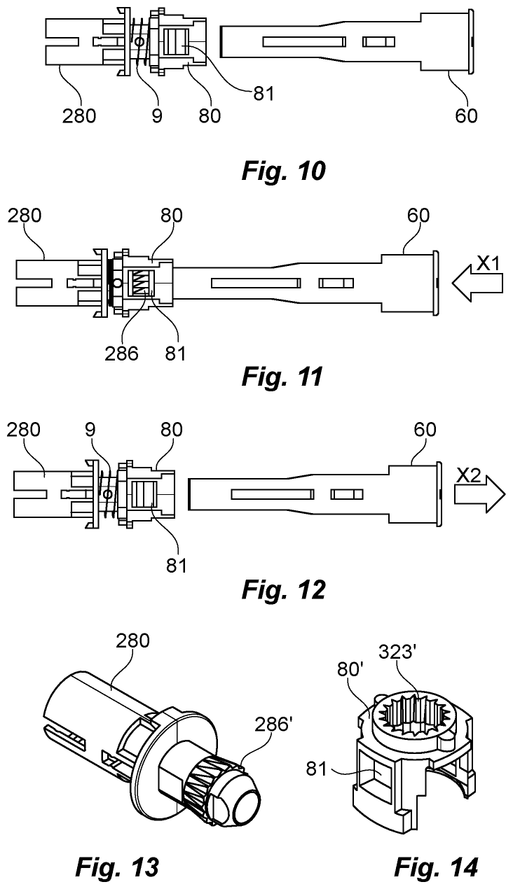

The pause mechanism provides a resilient member 9. The resilient member 9 is positioned between the driver 280 and the actuator sleeve 80, as shown in FIGS. 10-12. In this example, when the delivery member cover 60 is released from being pushed into the housing 40, meaning that the delivery member cover 60 is lifted from the medicament delivery site, the resilient member 9 moves the actuator sleeve 80 back to the first position. As a result, the blocking structure 323; 323′ re-engages with the counter-blocking structure 286 of the driver 280. Thus, if the injection is not completed, the engagement between the blocking structure 323; 323′ and the counter blocking structure 286 of the driver 280 causes the rotation of the driver 280 to stop. Therefore, the residual medicament will not be emptied, e.g., in the air, thus, the user and/or medical professionals can understand, from the amount of the residual medicament, about what dosage the user has been taken.

In one example where the actuator sleeve 80 is an integral part to the delivery member cover 60 or is axially immovable relative to the delivery member cover 60, the first resilient member configured to move the delivery member cover from the retracted position to the extended position can be the resilient member for causing the blocking structure to re-engage the counter blocking structure.

In a preferred example, the protrusions 286′ of the driver 280 can be further protruded and/or have greater inclination angles relative to the longitudinal axis 20, e.g., 50-70 degrees, such that the driver 280 can be easier stop by the re-engagement between the blocking structure and the counter blocking structure.

The resilient member 9 is configured to be tensioned by the actuator sleeve 80 when the actuator sleeve 80 is moved from the first position to the second position. In a preferred example, the resilient member 9 is a compression spring longitudinally extending between the driver 280 and the actuator sleeve 80, as shown in FIGS. 10-12. Alternatively, instead of extending from the driver, the resilient member can extend from the housing to the actuator sleeve 80. Alternatively, the resilient member can be a tension spring. Furthermore, as the resilient member 9 is configured to position the actuator sleeve 80 in the first position where the blocking structure is engaged with the protrusion of the driver 280, an accidental movement of the actuator sleeve 80 from the first position to the second position, e.g., when the medicament delivery device is dropped, can be prevented.

In one example where the blocking structure is provided on a flexible arm 324 of the blocking member 320, preferably, the flexible arm comprises a surface facing towards the actuator sleeve and the actuator sleeve comprises a counter surface facing towards the flexible arm. At least one of the surface and the counter surface is a chamfered surface such that when the counter surface of the actuator sleeve moves along the surface of the flexible arm, the flexible arm is bent radially towards the drive 280. The chamfered surface can facilitate the actuator sleeve to push the flexible arm towards the driver 280 thus, facilitating the blocking structure to re-engage with the protrusions of the driver 280 when the actuator sleeve 80 is moved from the second position to the first position.

In one example where the actuator sleeve is configured to rotate between the first position and the second position, the resilient member can be a torsion spring configured to rotate the actuator sleeve from the second position to the first position.

As mentioned above, it is desirable to keep the residual medicament in a used medicament delivery device so that the medicament intake of the user can be correctly observed and/or recorded. To facilitate the user and/or the medical professionals to observe the intake of the user, the pause mechanism of the drive assembly optionally comprises a residual dose indication. In a preferred example, the drive assembly comprises the housing 40; 40′; 40″ of the medicament delivery device.

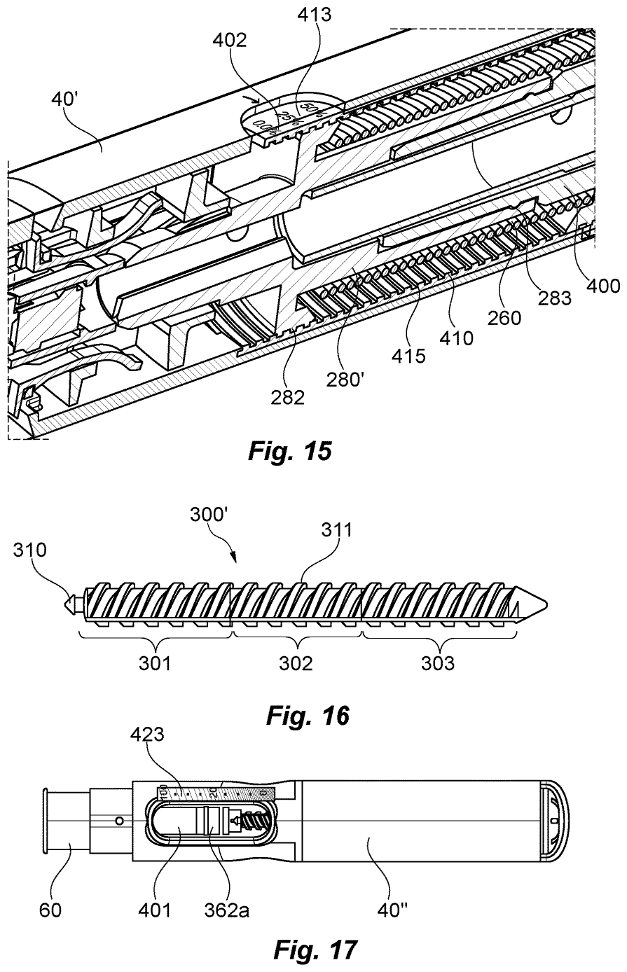

The residual dose indication comprises an axially extending scale indication 413; 301, 302, 303; 423 configured to be mechanically connected to the driver 280; 280′ and at least partially observable from outside of the housing 40; 40′; 40″ of the medicament delivery device such that the residual dose of the medicament of the medicament delivery device can be shown by the extending scale indication 413; 301, 302, 303; 423 to a user of the medicament delivery device when the driver 280; 280′ is stopped rotating. In one example, the axially extending scale indication is connected to the driver 280′ via a thread connection, as shown in FIG. 15. In this example, the axially extending scale indication is provided on a component that is directly and threadedly connected to the driver 280′. Alternatively, or additionally, the axially extending scale indication is provided on the outer surface of the plunger rod 300. Thus, the axially extending scale indication is connected to the driver 280 via the plunger rod 300. Alternatively, or additionally, the axially extending scale indication is provided on the housing 40″ of the medicament delivery device, thus, in this example, the axially extending scale indication is either connected to the driver 280 by the housing or via the inner housing. As how many rotations the driver has rotated can determine the residual medicament when the driver stops rotating, thus, the mechanical connection between the axially extending scale indication enables the axially extending scale indication to show the information about the residual medicament to the user.

In one example, as shown in FIG. 15, the housing 40′ comprises a second window 402 and an indication drum 410 is provided. The indication drum comprises the axially extending scale indication 413 configured to be partially lined up with the second window 402 of the housing 40′ such that the user can observe the scale indication 413 from the second window. The scale indication 413 can be formed by at least one of color marks, numerals, textures, letters, geometric marks, and a combination thereof. The indication drum 410 comprises a thread 415 configured to engage with a counter thread 282 of the driver 280′. In one example where the driver 280′ comprises the flange, the counter thread 282 is arranged on an outer surface of the flange that is facing towards the inner surface of the housing 40′. In this example, the housing is configured to limit the rotation of the indication drum 410. For example, the housing comprises a rib extending in the direction of the longitudinal axis 20 and positioned within a groove of the indication drum; alternatively, the housing and the indication drum form a non-circular interface observed along the longitudinal axis 20. As the driver 280′ is axially immovable, due to the engagement 283 with the inner housing 400, for example, the rotation of the driver 280′ moves the indication drum 410 axially via the engagement between the thread 415 and the counter thread 282. The axial movement of the indication drum 410 stops once the driver 280′ is stopped from rotating, e.g., the contained medicament is emptied or the resilient member 9 moves the actuator sleeve 80′ to the first position to lock the rotation of the indication drum 410 with the engagement of the blocking structure and the counter blocking structure. As a result, the amount of the residual medicament can be presented by the scale of the indication drum 410 that is lined up with the second window 402 of the housing 40′.

Alternatively, or additionally, the residual dose indication can be provided by the plunger rod 300′. In this example, the plunger rod 300′ comprises a plunger rod head 310 and a plunger rod body 311 axially extending from the plunger rod head. The plunger rod head 310 is configured to be operably engaged with a medicament container of the medicament delivery device; and the plunger rod body 311 comprises the axially extending scale indication 301, 302, 303, as shown in FIG. 16. Similarly, the axially extending scale indication is formed by at least one of color marks, numerals, textures, letters, and geometric marks, a combination thereof. In this example, the housing does not need the second window as the user/medical professionals can observe the scale indication from the main window 401 of the housing 40′. In a preferred example, the scale indication is formed by different color marks, preferably a series color marks from the different color gamuts.

Alternatively, or additionally, the residual dose indication can be provided by the housing 40″ providing the axially extending scale indication 423 adjacent and along the main window 401. In this example, the user/medical professionals can observe the residual medicament by comparing the position of the plunger rod head 310 or the stopper 362a of the medicament container 362. Similarly, the axially extending scale indication 423 s formed by at least one of color marks, numerals, textures, letters, geometric marks, and a combination thereof.

Furthermore, a delivery member cover lockout mechanism can be provided. The delivery member cover lockout mechanism is configured to block the delivery member cover 60 from being moved towards the distal end of the housing once the user lifts the medicament delivery device from the medicament delivery site. In one example, the delivery member cover lockout mechanism can be provided by a locking member of the delivery member cover and a counter locking member of the housing. In one example, one of the locking member and the counter locking member is a flexible tongue; and the other one of the locking member and the counter locking member is a ridge protrusion. The flexible tongue comprises a chamfered surface such that when the delivery member cover is moved towards the proximal end of the housing, the flexible tongue can be moved past the ridge protrusion. In one example where the delivery member cover comprises the flexible tongue, the flexible tongue comprises a distally directed surface configured to be engaged with a proximally directed surface of the ridge protrusion once the flexible tongue moves past the ridge protrusion such that the delivery member cover is blocked from moving towards the distal end of the housing.

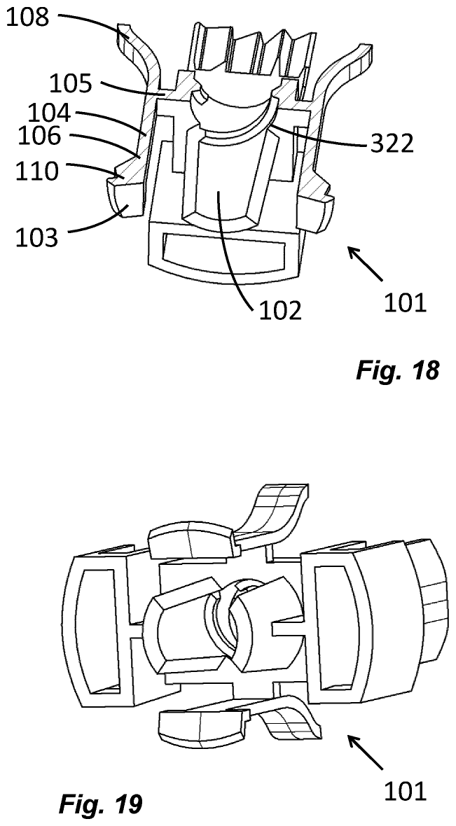

In another example, as shown in FIGS. 18-19, a lockout lever 104 is provided for the delivery member cover lockout mechanism. In this example, the lockout lever 104 comprises a fixed section 105 attached to the housing; a distal arm 108 extending from the fixed section 105 towards the distal end of the housing; and a proximal arm 106 extending towards the proximal end of the housing. The distal arm 108 and the proximal arm 106 are hinged to the fixed section 105 such that the distal arm 108 tilts radial outwardly from the axis 20 when the proximal arm 106 is pressed radial inwardly towards the axis 20; and the proximal arm 106 tilts radial outwardly from the axis 20 when the distal arm 108 is pressed radial inwardly towards the axis 20.

In a preferred example, the lockout lever 104 is attached to a thread insert 102. In this example, the thread insert 102 comprises a thread 322 configured to engage with the plunger rod 300; 300′ so that the plunger rod 300; 300′ can move axially when the driver 280; 280′ is rotating as mentioned above. The thread insert 102 is attached to the housing. In a preferred example, the proximal arm 106 of the lockout lever 104 is adjacent to a distally directed surface of the delivery member cover. In this example, the actuator sleeve 80; 80′ is initially blocked from moving in the proximal direction by a distally directed surface 110 of the lockout lever 104. In a preferred example, the distally directed surface 110 radially protrudes from the proximal arm 106.

As mentioned above, when the delivery member cover moves towards the distal end of the housing to the retracted position, the delivery member cover moves the actuator sleeve 80; 80′ from the first position to the second position. In one example where the lockout lever 104 is provided, the delivery member cover is configured to flex the proximal arm 106 of the lockout lever when the delivery member cover moves to the retraction position. As a result, the distally directed surface 110 is disengaged from the actuator sleeve 80; 80′. Thus, the actuator sleeve 80; 80′ can be moved further in the proximal direction when the actuator sleeve 80; 80′ is moved from the second position to the first position. In this example, the distal arm 108 of the lockout lever 104 is radially positioned inside the actuator sleeve 80; 80′. In this example, the actuator sleeve 80; 80′ comprises an inner protrusion arranged at the distal end of the actuator sleeve 80; 80′. Thus, when the actuator sleeve 80; 80′ moved further in the proximal direction, the inner protrusion of the actuator sleeve 80; 80′ can press the distal arm 108 of the lockout lever 104 inwardly towards the axis 20. As a result, the proximal arm 106 tilts radial outwardly from the axis 20. When the proximal arm 106 tilts radial outwardly from the axis 20, a proximally directed surface 103 of the proximal arm 106 blocks any further distal movement of the delivery member cover. The delivery member cover lockout mechanism is thus provided.

It should be noted that, the first position of the actuator sleeve is defined as a position that the blocking structure will positioned radially between the actuator sleeve and the driver; as the blocking structure can further extend in the longitudinal direction, in one example where the lockout lever 104 is provided, the initial position of the actuator sleeve and the position to which the actuator sleeve is proximally moved by the resilient member are both the first position of the actuator sleeve.

It should be noted that, the delivery member cover lockout mechanism might be essential from medical device regulatory aspect; however, the delivery member cover lockout mechanism is optional for the pause mechanism for a drive assembly of a medicament delivery device as disclosed above as the driver can be stopped from rotating as disclosed above with or without a delivery member cover lockout mechanism is provided. Similarly, the delivery member cover lockout mechanism is also optional for the medicament delivery device as disclosed above, as the medicament can be delivered with or without the delivery member cover being locked after used.

The inventive concept has mainly been described above with reference to a few examples. However, as is readily appreciated by a person skilled in the art, other embodiments than the ones disclosed above are equally possible within the scope of the inventive concept, as defined by the appended claims.

Some other aspects of the invention are defined by the following clauses.
1. A pause mechanism for a drive assembly of a medicament delivery device, the drive assembly comprising a driver (280; 280′) configured to be rotationally driven by a power source, an actuator sleeve (80; 80′), a blocking structure (323) positioned radially between the actuator sleeve (80; 80′) and the driver when the actuator sleeve is in the first position, wherein the blocking structure (323) is configured to engage with the driver (280; 280′) when the actuator sleeve is in the first position such that the rotation of the driver (280; 280′) relative to the blocking structure (323) is prevented, wherein when the actuator sleeve (80; 80′) is moved from the first position towards the second position the actuator sleeve (80; 80′) is configured to release the blocking structure (323) from the driver (280; 280′), enabling the driver (280; 280′) to rotate; wherein the pause mechanism comprises a resilient member (9) operably connected to the actuator sleeve (80; 80′) and is configured to be tensioned by the actuator sleeve (80; 80′) when the actuator sleeve (80; 80′) is moved from the first position to the second position; and wherein when the actuator sleeve (80; 80′) is moved from the second position to the first position by the resilient member (9) the blocking structure (323) is re-engaged with the driver (280; 280′).
2. The pause mechanism as described in clause 1, wherein the drive assembly comprises a delivery member cover (60) configured to be moved axially relative to the driver (280; 280′), between an extended position and a retracted position, wherein the delivery member cover (60) is configured to move the actuator sleeve (80; 80′) from a first position to a second position when the delivery member cover is moved from the extended position to the retracted position.
3. The pause mechanism as described in clause 2, wherein the actuator sleeve is axially immovable relative to the delivery member cover.
4. The pause mechanism as described in any of the preceding clauses, wherein the resilient member is longitudinally extending between the actuator sleeve and the driver; wherein the resilient member is compressed when the actuator sleeve is moved from the first position to the second position.
5. The pause mechanism as described in any of the preceding clauses, wherein the pause mechanism comprises a residual dose indication; wherein the residual dose indication comprises an axially extending scale indication (413; 301, 302, 303; 423) configured to be mechanically connected to the driver (280; 280′) and at least partially observable from an outside of a housing of the medicament delivery device such that the residual dose of the medicament of the medicament delivery device can be shown by the extending scale indication (413; 301, 302, 303; 423) to a user of the medicament delivery device when the driver (280; 280′) is stopped rotating.
6. The pause mechanism as described in clause 5, wherein the residual dose indication comprises an indication drum (410) having the axially extending scale indication; wherein the indication drum (410) comprises a thread (415) engaged with a counter thread (282) of the driver (280′) such that the rotation of the driver (280′) coverts to an axial movement of the indication drum (410) via the engagement between the thread (415) and the counter thread (282).
7. The pause mechanism as described in any of the preceding clauses, wherein the drive assembly comprises a plunger rod (19; 19′) operably engaged with the driver such that the driver moves the plunger rod axially when the driver is rotated.
8. The pause mechanism as described in clause 7, wherein the plunger rod is rotationally engaged with the driver such that the plunger rod is rotated by the driver when the driver is rotated.
9. The pause mechanism as described in clause 7 or 8 when dependent on clause 5, wherein the plunger rod comprises a plunger rod head and a plunger rod body axially extending from the plunger rod head; wherein the plunger rod head is configured to be operably engaged with a medicament container of the medicament delivery device; and wherein the residual dose indication is provided with the plunger rod body comprises the axially extending scale indication.
10. The pause mechanism as described in clause 5, wherein the axial extending scale indication is positioned on a housing of the medicament delivery device; and wherein the axial extending scale indication is position adjacent and along a main window of the housing of the medicament delivery device.
11. The pause mechanism as described in any of the preceding clauses when dependent on clause 5, wherein the axially extending scale indication is formed by at least one of color marks, numerals, textures, letters, geometric marks, and a combination thereof.
12. A drive assembly comprising the pause mechanism as described in any of the preceding clauses, the drive assembly comprising
   a driver configured to be rotationally driven by a power source, an actuator sleeve, a blocking structure positioned radially between the actuator sleeve and the driver when the actuator sleeve is in a first position, wherein the blocking structure is configured to engage with the driver when the actuator sleeve is in the first position such that the rotation of the driver relative to the blocking structure is prevented, and wherein when the actuator sleeve is moved from the first position towards a second position the actuator sleeve is configured to release the blocking structure from the driver, enabling the driver to rotate.
13. The drive assembly as described in clause 12, comprising a delivery member cover configured to be moved axially relative to the driver between an extended position and a retracted position, wherein the delivery member cover is configured to move the actuator sleeve from a first position to a second position when the delivery member cover is moved from the extended position to the retracted position.
14. The drive assembly as described in clauses 12 or 13, wherein the driver comprises a counter-blocking structure configured to be engaged with the blocking structure when the actuator sleeve is in the first position.
15. The drive assembly as described in clause 14, wherein the blocking structure comprises a protrusion extending radially towards the driver; and the counter-blocking structure of the driver comprises protrusions radially extending towards the blocking structure; and wherein the protrusion of the blocking structure is configured to engage with the protrusions when the actuator sleeve is in the first position.
16. The drive assembly as described in any one of clauses 12-15, comprising a blocking member; wherein the blocking member comprises a flexible arm being radially flexible; wherein the blocking structure is arranged on a surface of the flexible arm facing towards the driver; and wherein the actuator sleeve is configured to enable the flexible arm to flex radially outwards when the actuator sleeve is in the second position.
17. The drive assembly as described in clause 16, wherein the actuator sleeve comprises a support wall; wherein the flexible arm is positioned radially between the driver and the support wall of the actuator sleeve when the actuator sleeve is in the first position such that the flexible arm is prevented from flexing radially outward; and wherein the support wall is axially offset from the flexible arm when the actuator sleeve is in the second position such that the flexible arm is able to flex radially outwards.
18. The drive assembly as described in clauses 16 or 17, wherein the blocking member is ring-shaped.
19. The drive assembly as described in any one of clauses 16-18, wherein the driver is configured to provide a rotational force on the flexible arms, urging the flexible arms radially outwards.
20. The drive assembly as described in any one of clauses 16-19, wherein the flexible arm comprises a surface facing towards the actuator sleeve in the direction of the longitudinal axis; wherein the actuator sleeve comprises a counter surface facing towards the flexible arm in the direction of the longitudinal axis; and wherein at least one of the surface and the counter surface is a chamfered surface such that when the counter surface of the actuator sleeve moves along the surface of the flexible arm, the flexible arm is bent radially towards the drive.
21. The drive assembly as described in clause 20, wherein the flexible arm comprises a free end; and wherein the surface of the flexible arm facing towards the actuator sleeve is located at the free end of the flexible arm.
22. The drive assembly as described in any one of clauses 12-15, wherein the blocking structure is an integral part of the actuator sleeve extending from the actuator sleeve towards the driver; wherein the blocking structure is disengaged from the driver when the actuator sleeve is in the second position.

1-14. (canceled)

15. A drive assembly of a medicament delivery device, the drive assembly comprising:
a driver configured to be rotationally driven by a power source;
an actuator sleeve; and
a blocking structure positioned radially between the actuator sleeve and the driver when the actuator sleeve is in a first position, wherein the blocking structure is configured to engage with the driver when the actuator sleeve is in the first position such that the rotation of the driver relative to the blocking structure is prevented,
wherein when the actuator sleeve is moved from the first position towards a second position the actuator sleeve is configured to release the blocking structure from the driver, enabling the driver to rotate,
wherein the drive assembly further comprises a resilient member operably connected to the actuator sleeve and is configured to be tensioned by the actuator sleeve when the actuator sleeve is moved from the first position to the second position, and
wherein when the actuator sleeve is moved from the second position to the first position by the resilient member the blocking structure is re-engaged with the driver.

16. The drive assembly of claim 15, wherein the drive assembly comprises a delivery member cover configured to be moved axially relative to the driver, between an extended position and a retracted position, wherein the delivery member cover is configured to move the actuator sleeve from a first position to a second position when the delivery member cover is moved from the extended position to the retracted position.

17. The drive assembly of claim 16, wherein the actuator sleeve is axially immovable relative to the delivery member cover.

18. The drive assembly of claim 15, wherein the resilient member is longitudinally extending between the actuator sleeve and the driver, wherein the resilient member is compressed when the actuator sleeve is moved from the first position to the second position.

19. The drive assembly of claim 15, wherein the pause mechanism comprises a residual dose indication, wherein the residual dose indication comprises an axially extending scale indication configured to be mechanically connected to the driver and at least partially observable from an outside of a housing of the medicament delivery device such that the residual dose of the medicament of the medicament delivery device can be shown by the extending scale indication to a user of the medicament delivery device when the driver is stopped rotating.

20. The drive assembly of claim 19, wherein the residual dose indication comprises an indication drum having the axially extending scale indication, wherein the indication drum comprises a thread engaged with a counter thread of the driver such that the rotation of the driver coverts to an axial movement of the indication drum via the engagement between the thread and the counter thread.

21. The drive assembly of claim 19, wherein the drive assembly comprises a plunger rod operably engaged with the driver such that the driver moves the plunger rod axially when the driver is rotated.

22. The drive assembly of claim 21, wherein the plunger rod is rotationally engaged with the driver such that the plunger rod is rotated by the driver when the driver is rotated.

23. The drive assembly of claim 21, wherein the plunger rod comprises a plunger rod head and a plunger rod body axially extending from the plunger rod head, wherein the plunger rod head is configured to be operably engaged with a medicament container of the medicament delivery device, and wherein the residual dose indication is provided with the plunger rod body comprises the axially extending scale indication.

24. The drive assembly of claim 19, wherein the axially extending scale indication is formed by at least one of color marks, numerals, textures, letters, geometric marks, and a combination thereof.

25. The drive assembly of claim 15, further comprising a delivery member cover configured to be moved axially relative to the driver between an extended position and a retracted position, wherein the delivery member cover is configured to move the actuator sleeve from a first position to a second position when the delivery member cover is moved from the extended position to the retracted position.

26. The drive assembly of claim 15, wherein the driver comprises a counter-blocking structure configured to be engaged with the blocking structure when the actuator sleeve is in the first position.

27. The drive assembly of claim 26, wherein the blocking structure comprises a protrusion extending radially towards the driver and the counter-blocking structure of the driver comprises protrusions radially extending towards the blocking structure, and wherein the protrusion of the blocking structure is configured to engage with the protrusions when the actuator sleeve is in the first position.

28. The drive assembly of claim 15, further comprising a blocking member, wherein the blocking member comprises a flexible arm being radially flexible, wherein the blocking structure is arranged on a surface of the flexible arm facing towards the driver, and wherein the actuator sleeve comprises one or two cut-outs to be aligned with the flexible arm when the actuator sleeve is in the second position such that the flexible arm can flex radially outwards.
